SEQUENCE LISTING (5 sequences)

SEQ ID NO: 1 - DNA, 930 bp, Sus scrofa
atggttggattcaaggccacagaggtccccccgactgccactgtgaagttcctgggggctggcacagctgcctgcatcgcagacctcatcacctttcccctggacacggctaaagtccggctgcagatccagggagaaaggcgggggccagtgcaggccgcggccagtgcccagtaccgcggggtgctgggcaccattctcaccatggtgcgcaacgagggcccccgcagcctctacaacgggctggtggccggcctgcagcgccagatgagcttcgcctccgtccgcatcggcctctacgactccgtcaagcatttctacaccaagggctcagagcatgctggcatcgggagccgcctcctggcaggcagcaccacgggggccttggctgtggccgtggcccagccaacagacgtggtaaaggtccggttccaagcgcaggcccgggccggcggaggccggcggtaccggagcactgtcgacgcctacaagaccatcgcccgagaggaggggctgcggggcctctggaaagggacctcacccaatgtcgctcgtaatgccattgtcaactgtgctgagctggtgacctatgacctcatcaaggacacgctcctgaaggccgacctcatgacagatgaccttccctgccacttcacgtccgccttcggggcgggcttctgcaccaccgtcatcgcctctcccgtggacgtggtcaagacgagatacatgaactctgccccgggccagtacagcagcgctggccactgtgccctcaccatgctccagaaggagggtccccgagccttctacaaagggttcacgccctcctttctccgattggggtcctggaacgtggtgatgtttgtcacctatgagcagctgaagagggccctcatggctgcccgcgcttcccgagaggctcccttttga

SEQ ID NO: 2 - DNA, 930 bp, Sus scrofa
atggttggattcaaggccacagaggtccccccgactgccactgtgaagttcctgggggctggcacagctgcctgcatcgcagacctcatcacctttcccctggatacggctaaagtccggctgcagatccagggagaaaggcgggggccagtgcaggccgcggccagtgcccagtaccgcggggtgctgggcaccattctcaccatggtgcgcaacgagggcccccgcagcctctacaacgggctggtggccggcctgcagcgccagatgagcttcgcctccgtccgcatcggcctctacgactccgtcaagcatttctacaccaagggctcagagcatgctggcatcgggagccgcctcctggcaggcagcaccacgggggccttggctgtggccgtggcccagccaacagacgtggtaaaggtccggttccaagcgcaggcccgggccggcggaggccggcggtaccggagcactgtcgacgcctacaagaccatcgcccgagaggaggggctgcggggcctctggaaagggacctcacccaatgtcgctcgtaatgccattgtcaactgtgctgagctggtgacctatgacctcatcaaggacacgctcctgaaggccgacctcatgacagatgaccttccctgccacttcacgtccgccttcggggcgggcttctgcaccaccgtcatcgcctctcccgtggacgtggtcaagacgagatacatgaactctgccccgggccagtacagcagcgctggccactgtgccctcaccatgctccagaaggagggtccccgagccttctacaaagggttcacgccctcctttctccgattggggtcctggaacgtggtgatgtttgtcacctatgagcagctgaagagggccctcatggctgcccgcgcttcccgagaggctcccttttga

SEQ ID NO: 3 - DNA, 24 bp, Artificial Sequence
  FLAG sequence
gactacaaggacgatgatgacaag

SEQ ID NO: 4 - DNA, 21 bp, Artificial Sequence
  Forward Primer
ccaagcaaagagaggcactca

SEQ ID NO: 5 - DNA, 21 bp, Artificial Sequence
  Reverse Primer
ctaactggtctttgatggcgg

(19) United States
(12) Patent Application Publication
(10) Pub. No.: US 2021/0164019 A1
(43) Pub. Date: Jun. 3, 2021

(54) IDENTIFICATION, FUNCTION AND APPLICATION OF M6A METHYLATION SITE IN PIG FAT DEPOSITION-RELATED FAM134B MRNA

(71) Applicant: Zhejiang University, Hangzhou (CN)

(72) Inventors: Yizhen Wang, Hangzhou (CN); Xinxia Wang, Hangzhou (CN); Min Cai, Hangzhou (CN); Fengqin Wang, Hangzhou (CN)

(21) Appl. No.: 16/701,142

(22) Filed: Dec. 2, 2019

(51) Int. Cl. C12Q 1/686; C12Q 1/6851; C12Q 1/6804

(52) U.S. Cl. CPC C12Q 1/686; C12Q 2600/154; C12Q 1/6804; C12Q 1/6851

(57) ABSTRACT

The identification, function and application of m⁶A methylation site in the FAM134B mRNA. The main steps include confirming the m⁶A methylation site in FAM134B mRNA by comparative analyzing m⁶A-seq results of Landrace and Jinhua pigs, corresponding with highly conserved motif RRACH (R=G, A; H=A, C, T) and the prediction website; altering the m⁶A methylation in FAM134B mRNA via mutating synonymous codon of FAM134B gene (C1358 to T1358) without changing the amino acid sequence; designing qPCR primers according to the m⁶A peak region and a control region of FAM134B mRNA; extracting total RNA and determining the relative m⁶A level of a single gene by protein immunoprecipitation and qPCR. The m⁶A methylation site of FAM134B mRNA plays a critical rule on fat deposition, which serves as a novel molecular marker and a drug target for treating obesity.

### FIELD OF THE INVENTION

The present invention relates to the field of molecular biology, and is connected with the identification, function and application of m⁶A methylation site in a single gene mRNA. Specifically, it is correlated with the identification, function and application of the m⁶A methylation site in FAM134B mRNA.

### BACKGROUND OF THE INVENTION

Along with the development of social economy and scientific technological level, the living standards of people is improved. The meat products, especially pork products, take a big proportion of food consumption in china. However, the quality of pork cannot fully meet consumer's requirements currently. The fatty acid content of pork is influenced by many factors such as dietary components, feeding methods, breeds and age of pigs. Most local pig breeds in China have high backfat thickness and intramuscular fat content, while the taste of local pig breeds also better than foreign and cross-bred breeds. With the rate of the foreign genomes constitute in crossing pig breeds increasing, the quality of pork becomes reducing.

RNA post-translational modification which contained 80% methylation modification, establishes the chemistry foundation of diversity RNA function. N6-methyladenosine m⁶A, which refers to the methylation of the adenosine nucleotide acid at the nitrogen-6 position, is the most prevalent post-transcriptional modification of eukaryotic mRNA and receives extensive attention and researches in recent years. m⁶A RNA methylation modification, which is discovered in bacteria DNA initially, is detected in varied high eukaryotes and virus. Since the existence of m⁶A RNA methylation is so widespread, it is difficult to ignore its biological significance and importance. The m⁶A methylation site mainly exists in the highly conserved sequence, RRACH (R=G, A; H=A, C, T), and may plays a fatal role in epigenetic regulation. According to the high-throughput sequencing technology, the crude map of m⁶A modification has been screened.

In recent years, it has been explored that FAM134B plays a crucial part in adipogenesis. Depending on the previous data of m⁶A-seq, it has been revealed that the m⁶A methylation content of FAM134B mRNA has difference in fat and lean pigs. Consequently, it is necessary to recognize the m⁶A methylation site in FAM134B mRNA and provide a new molecular marker for the enhancement of genetic traits in pig fat deposition by identifying the biological functions.

### SUMMARY OF THE INVENTION

The invention is to offer a method for recognizing the m⁶A methylation site in FAM134B mRNA which modulates pig fat deposition.

The invention is to provide a method for measuring the relative difference in m⁶A level of FAM134B mRNA.

The invention is to offer a method for changing the m⁶A content of FAM134B mRNA at the gene level.

The invention is to demonstrate the function of the m⁶A methylation site in FAM134B mRNA which regulates adipogenesis.

The invention is to demonstrate the specific m⁶A methylation site by analyzing bioinformation according to the conserved m⁶A motif.

The invention offers primer sequences and specific methods for testing the m⁶A level of FAM134B mRNA in pigs.

The present invention identifies the role and function of the m⁶A methylation site in FAM134B mRNA during fat deposition by changing the m⁶A content of FAM134B mRNA via single mutation.

The present invention demonstrates that the m⁶A methylation site of FAM134B mRNA plays a fatal effect on fat deposition, which can provide a novel molecular marker and a drug target for treating obesity, and also a beneficial genetic resource for molecular breeding or transgene of pigs.

### BRIEF DESCRIPTION OF THE DRAWINGS

FIG. 1: The difference in the site and level of m⁶A methylation which are in FAM134B mRNA between layer of backfat of Landrace pigs (L-LB) and Jinhua pig (J-LB).

FIG. 2: Synonymous mutation in FAM134B mRNA which changes the m⁶A level.

FIG. 3: The alteration in m⁶A level of FAM134B mRNA after m⁶A site mutation.

FIG. 4: The effect of m⁶A site mutation on adipogenesis.

FIG. 5: The effect of m⁶A site mutation on protein expression of FAM134B.

FIG. 6: The effect of m⁶A site mutation on mRNA stability of FAM134B.

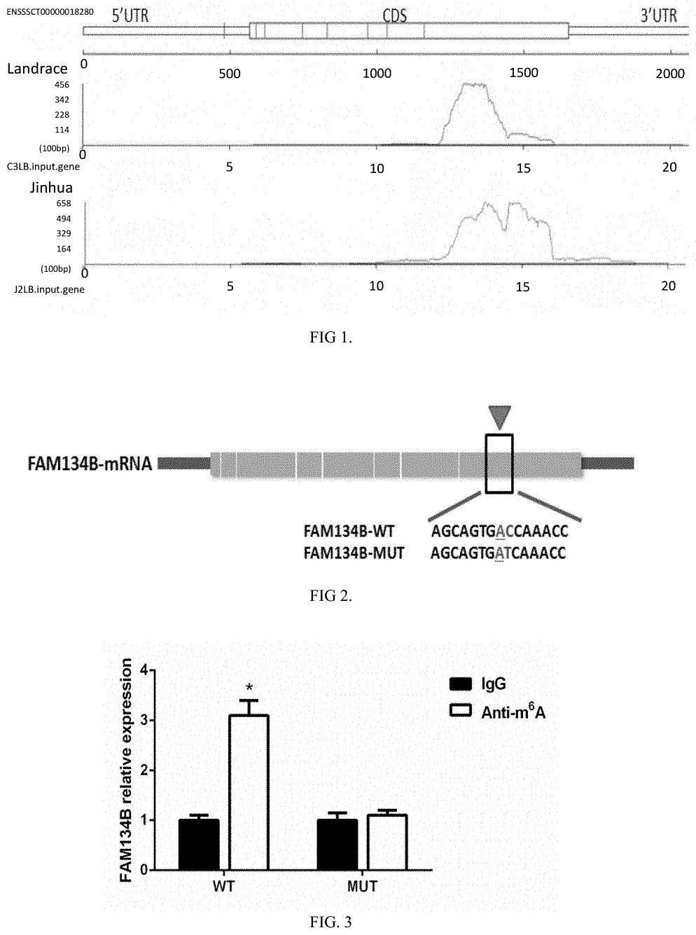

### DETAILED DESCRIPTION OF THE INVENTION

Embodiment 1: The different content of m⁶A methylation in FAM134B mRNA between Landrace pigs (L-LB) and Jinhua pigs (J-LB).

The RNA which is used in the experiment is derived from the adipose tissue of Landrace pigs and Jinhua pigs. The following is the specific Embodiments:

1. Extraction of total RNA from adipose tissue of pigs

The total RNA of pig adipose tissue is extracted by Trizol (conventional methods). The specific method is as follows:
1) Take the adipose tissue sample frozen in liquid nitrogen, grind 50-100 mg of adipose tissue into powder in liquid nitrogen, and place it in RNase-free 1.5 ml microcentrifuge tube (EPPENDORF TUBES).
2) Add 1 ml of Trizol and shake vigorously.
3) Add 200 μl of chloroform, shake vigorously and centrifuge at 12000 g for 15 min at 4° C.
4) Transfer the supernatant into another RNase-free 1.5 ml microcentrifuge tube (EPPENDORF TUBES), add equal volume of isopropanol, incubate for 10 min at room temperature with gentle mixing, and centrifuge at 12000 g for 15 min at 4° C.
5) Remove the supernatant, wash the pellet with 1 ml of 75% ethanol, centrifuge at 7500 g for 5 min at 4° C.

6) Dissolve the pellet with 20-50 μl of RNase-free water.

2. mRNA elution

mRNA was eluted using GENELUTE mRNA miniprep Kits (Sigma). The specific method is as follows:
1) Expand the volume of total RNA sample to 250 μl add 250 pi binding solution and mix.
2) Add 15 μl of beads, shake vigorously and incubate at 70° C. for 3 min, then place at room temperature for 10 min.
3) Centrifuge the beads-lysate mixture at maximum speed for 2 min and discard the supernatant.
4) Beads were resuspended with 500 μl wash buffer and transferred to the spin filter.

5) Centrifuge for 1-2 min and discard the flow through.
6) Repeat procedure 4) and 5).
7) Add 50 μlof Elution Buffer heated to 70° C. and incubate for 2-5 min at 70° C.
8) Centrifugal and harvest mRNA.
9) Vacuum concentration the mRNA to 9 μl.

3. mRNA fragmentation
Fragmentation reaction

| | Volume per |
|---|---|
| Component | sample |
| RNA(1 μg/μ1) | 9 |
| Fragmentation buffer (10×) | 1 |
| Total volume | 10 |

70° C., 15 min, add EDTA to stop reaction,
Fragment Buffer (10×): 800 μl RNase-free water, 100 μl 1 M Tris-HCl (pH=7.0), 100 μl 1 M ZnCl₂.

4. Detection of mRNA Fragmentation

The fragmented sample is subjected to 1×TAE electrophoresis on a 2% (wt/vol) agarose gel to detect the fragment size. The fragment is 100-200 bp, which is appropriately sized and needed to be concentrated to 1 μg/100 μl for subsequent experiments.

5. Immunoprecipitation
1) Reagents preparation
10% Igepal CA-630 (Sigma-Aldrich, cat. no. I8896)

| | | | Final |
|---|---|---|---|
| Component | Stock | Amount | concentration |
| Igepal CA-630 | 100% | 1 ml | 10% |
| RNAase free water | | 9 ml | |

5×IP buffer

| | | | Final |
|---|---|---|---|
| Component | Stock | Amount | concentration |
| Tris-HCl (pH 7.4) | 1M | 0.5 ml | 0.05M |
| NaCL | 5M | 1.5 ml | 0.75M |
| Igepal CA-630 | 10% | 0.5 ml | 0.5% |

| | |
|---|---|
| RNAase free water | To 10 ml final volume |

Freshly prepare 1×IP buffer

| | | | Final |
|---|---|---|---|
| Component | Stock | Amount | concentration |
| 5 × IP buffer | 5× | 1 ml | 1× |
| RNase inhibitors# | 20 U/μl | 50 μl | 200 U/ml |

| | |
|---|---|
| RNAase free water | To 5 ml final volume |

(#SUPERase• In ™ RNase Inhibitor (20 U/μl) Thermo Fisher (AM2696))

Elution buffer

| | | | | Final |
|---|---|---|---|---|
| Component | Stock | | Amount | concentration |
| 5 × IP buffer | 5× | | 90 μl | 1× |
| m⁶A# | 20 | mM | 150 μl | 6.7 mM |
| RNase inhibitors# | 20 | U/μl | 7 μl | 140 U |
| RNAase free water | 203 | μl | | |

(#N6-Methyladenosine, 50-monophosphate
sodium salt (m⁶A, Sigma-Aldrich, cat. no. M2780))
2) RNA Immunoprecipitation

The RNA fragment obtained from step 4 is then used to do the following experiments:

| | |
|---|---|
| mRNA fragmentation | 100 μl |
| 5 × IP buffer | 40 μl |
| RNAase inhibitor | 10 μl |
| m⁶A-antibody (0.5 mg/ml) | 5-8 μl |
| RNAase free water | 42-45 μl |
| Total | 200 μl |

4° C., 2 h, on rotation wheel
3) Beads blocking (Dynabeads® Protein A, Life technologies, 10002D)
Take beads 40 μl/sample, put it in magnetic stand and take supernatant out. Beads were wash three times in 1 ml 1×IP buffer and resuspended in 1 ml 1×IP blocking buffer.

| | |
|---|---|
| 5 × IP buffer | 200 μl |
| RNAase inhibitor | 10 μl |
| BSA | 25 μl |
| RNAase free water | 765 μl |
| Total | 1000 μl |

Incubate beads at 4° C. for 2 h. Beads were wash three times in 1 ml 1×IP buffer and resuspended in 100 μl 1×IP buffer.
4) Beads-m⁶A antibody-RNA complex
Mix the samples from 2) and 3), and incubate at 4° C. for 2 h.
5) Elution I
Take beads from 4) and put it on magnetic stand, discard supernatant.
Beads were wash three times in 500 μl 1×IP buffer and resuspended in 150 μl Elution buffer. Incubate beads at 4° C. for 1 h.

| | |
|---|---|
| 5 × IP buffer | 90 μl |
| RNAase inhibitor | 7 μl |
| m⁶A | 150 μl |
| RNAase free water | 203 μl |
| Total | 450 μl |

6) Elution II
Take beads from 5) and put it on magnetic stand, collect supernatant. Add 50 μl Elution buffer. Incubate beads at 4° C. for 30 min.
7) 1st and 2nd Elution combined to get 200 μl RNA solution
8) Ethanol precipitate
Add 20 μl NaOAc, 500 μl Ethanol (100%), 4 μl glycogen (5 mg/ml) to RNA solution and precipitate overnight.
9) The next day, centrifuge at 14000 rpm for 30 min at 4° C. Discard supernatant, keep pellet, add 1 ml 75% ethanol and centrifuge at 14000 rpm for 30 min at 4° C. Discard supernatant, keep pellet, add 9 μl RNase-free water and measure concentration.

6. Library preparation

Using TRUSEQ Stranded mRNA Library Prep Kit (Illumina).
1) First strand synthesis
Take remaining 6-7 μl of immunoprecipitated fragmented RNA, add 10-11 μl FPF mix, incubate at 94° C. for 10 s and place on ice immediately.
PCR procedure: 25° C. 10 min, 42° C., 15 min, 70° C., 15 min, Hold at 4° C.

| | |
|---|---|
| 17 μl mixture | 17 μl |
| First strand synthesis | 7.2 μl |
| Act D mix (FSA) | |
| Superscript II reverse | 0.8 μl |
| transcriptase | |
| Total | 25 μl |

(SUPERSCRIPT II reverse transcriptase invitrogen 18064-014)
2) Second strand synthesis
PCR procedure: 16° C. 1 h, then put on

| | |
|---|---|
| 25 μl product from 1) | 25 μl |
| Second strand marking master Mix | 20 μl |
| Resuspension buffer | 5 μl |
| Total | 50 μl |

3) Purify cDNA Using AMPure XP Beads
Add 90 μl AMPure XP beads to 50 μl cDNA (get from 2)), place at room temperature for 15 min. Put it on magnetic stand for 5 min, remove 135 μl supernatant, wash beads twice with 200 μl 80% ethanol, then remove ethanol completely. place at room temperature for 5 min. Then add 20 μl resuspension buffer at room temperature for 2 min. Put it on magnetic stand for 5 min, take 17.5 μl supernatant to new tube.
4) Adenylate 3′End
Add 12.5 μl A-tailing mix to 17.5 μl cDNA.
PCR procedure: 37° C., 30 min, 70° C., 5 min, Hold at 4° C.
5) Ligation Adapters

| | |
|---|---|
| dscDNA from 4) | 30 μl |
| Ligation mix | 2.5 μl |
| RNA adapter index | 2.5 μl |
| resuspension buffer | 2.5 μl |
| Total | 37.5 μl |

Add 5 μl stop ligation buffer (totally 42.5 μl).
6) 1st Clean Up the Above Product Using AMPURE XP Beads
Add 42 μl AMPURE XP beads and place at room temperature for 15 min. Put beads on magnetic stand, remove 79.5 μl supernatant. Wash beads twice with 200 μl 80% ethanol, then remove ethanol completely. Place at room temperature for 5 min to dry. Then add 52.5 μl resuspension buffer at room temperature for 2 min. Put it on magnetic stand for 5 min, take 50 μl supernatant to new tube.
7) 2nd Clean Up the Above Product Using AMPURE XP Beads
Add 50 μl AMPURE XP beads and place at room temperature for 15 min. Put beads on magnetic stand for 5 min, remove 95 μl supernatant. Wash beads twice with 200 μl 80% ethanol, then remove ethanol completely. Place at room temperature for 5 min to dry. Then add 22.5 μl resuspension buffer at room temperature for 2 min. Put it on magnetic stand for 5 min, take 20 μl supernatant to new tube.
8) PCR Enrichment

| | |
|---|---|
| 20 μl product from 7) | 20 μl |
| PCR primer cocktail | 5 μl |
| PCR master mix | 25 μl |
| Total | 50 μl |

PCR procedure: 98° C. 30 s; 98° C., 10 s, 60° C., 30 s, 72° C., 30 s, 13-15 cycles; 72° C., 5 min; Hold at 4° C.
9) Cleanup PCR Product
Add 50 μl AMPURE XP beads and place at room temperature for 15 min. Put beads on magnetic stand for 5 min, remove 95 μl supernatant. Wash beads twice with 200 μl 80% ethanol, then remove ethanol completely. Place at room temperature for 5 min to dry. Then add 32.5 μl resuspension buffer at room temperature for 2 min. Put it on magnetic stand for 5 min, take 20 μl supernatant to new tube. Measure concentration by Qubit.

7. Sequencing and bioinformatics analysis

1) Detection and analysis by Bioanalyzer.

2) High-throughput sequencing using illumina's HISEQ 4000 platform and bioinformatics analysis showed significant differences in m⁶A levels of FAM134B mRNA between Landrace and Jinhua pigs (FIG. 1).

3) According to the gene sequence and prediction website (http://www.cuilab.cn), the m⁶A in FAM134B mRNA is located at 1358 site.

Embodiment 2: The point mutation that change the m⁶A methylation level of FAM134B mRNA

(Note: Since the A site is located in the second position of the codon, in order to stabilize the amino acid sequence and achieve synonymous mutation, only the third position of the codon can be mutated. The A of m⁶A is sited in the conserved sequence GGACU, which contained an important C behind A that is necessary for m⁶A formation. The mutation of C will change the methylation efficiency of A, which decreases the m⁶A level, and successfully completes the aim of changing the m⁶A level.)

The porcine FAM134B gene (NM_001098605.1) sequence and the C1358 to T1358 mutation sequence were cloned. The FAM134B gene has the sequence of SEQ ID NO:1 and the FAM134B gene with mutation has the sequence of SEQ ID NO:2. The FLAG sequence (5′-GACTACAAGGACGATGATGACAAG-3′, SEQ ID NO:3) was added at the N-terminus.

These sequences were cloned into the HindIII and BamHI positions of the Pcdna3.1(+) expression plasmid to get the FAM134B-WT and FAM134B-MUT plasmids (the cloning process is a routine method, and the specific steps are omitted).

Embodiment 3: The change of m⁶A levels of FAM134B mRNA after point mutation

1. Primers Design

qPCR primers were design according to upstream and downstream of the mutation site, and synthesized by Sangon Biotech (China).

pFAM134B-m⁶A-F
SEQ ID NO: 4
5'-CCAAGCAAAGAGAGGCACTCA-3',
pFAM134B-m⁶A-R
SEQ ID NO: 5
5'-CTAACTGGTCTTTGATGGCGG-3',

2. Isolation and Culture of Porcine Preadipocytes

The method of isolation of porcine preadipocyte was based on published method (Ding et al., 1999 and Zhang et al., 2005) with minor modifications. Briefly, adipose tissue of 5-day-old Duroc-Landrace-Yorkshire piglets was isolated under sterile conditions and washed with high concentration of penicillin/streptomycin containing PBS. The visible blood vessels and muscles were removed. The adipose tissue was cut into pieces with scissors and placed in a sterile tube, digested by collagenase I (Gibco, USA) at 37° C. for 1 h. Add complete medium to stop digestion and filter digested tissue through 200 mesh and 300 mesh nylon net. Centrifuge at 1500 rpm for 10 min. Discard the supernatant, add the red blood cell lysate, squirt evenly, place at room temperature for 10 min. Centrifuge at 1500 rpm for 5 min, discard the supernatant, resuspend the cells in complete medium and transfer into 10 cm dish.

3. Porcine preadipocytes were transfected with FAM134B-WT or FAM134B-MUT plasmid

The transfections of FAM134B-WT and FAM134B-MUT plasmids were performed using Lipofectamine® 2000 (Invitrogen, USA) according to the manufacturers' instruction.

1) One day before transfection, the cells were trypsinized and counted, and cultured in complete medium without antibiotics. Seed cells to be 70-90% confluent at transfection.

2) Dilute 3 μg of DNA per well with 50 μl of OPTI-MEMI medium.

3) Dilute 10 μl of Lipofectamine 2000 Reagent with 50 μl OPTI-MEMI medium.

4) Add diluted DNA to diluted Lipofectamine® 2000 Reagent (1:1 ratio), incubated at room temperature for 5 min.

5) Add DNA-lipid complex directly to cells, gently shake the plate.

6) Change the medium after incubating cells for 4-6 h at 37° C.

7) After 24 h of transfection, the cells were collected.

4. Total RNA Extraction, Fragmentation, Immunoprecipitation and Reverse Transcription (as Above)

5. qPCR Analysis

| | |
|---|---|
| SYBR Green PCR | 5 μl |
| Master Mix | |
| Forward primer | 0.5 μl |
| Reverse primer | 0.5 μl |
| cDNA | 4 μl |
| Total | 10 μl |

qPCR procedure: 95° C. 2 min ; 95° C., 20s, 64° C., 20s, 72° C., 30s, 45 cycles. ATCB was used as internal control. The data were analysed following the 2^{-ΔΔCt} method. The calculation formula was as follows:

ΔΔCt=(Ct_{Target}−Ct_{Input})x−(Ct_{Target}−Ct_{Input})_{Control}

As shown in FIG. 3, the m⁶A level of FAM134B mRNA was changed after point mutation.

Embodiment 4: The Effect of m⁶A Level of FAM134B mRNA on Adipogenesis

1. Isolation and Culture of Porcine Preadipocytes (as Above)

2. Porcine Preadipocytes were Transfected with FAM134B-WT or FAM134B-MUT Plasmid (as Above)

3. Differentiation of Porcine Preadipocytes

After two-day post-confluence of cells, adipocyte differentiation was induced by adipogenic differentiation medium containing 0.5 mM IBMX (Sigma-Aldrich, I7018), 1 μM dexamethasone (Sigma-Aldrich, D1756) and 1 μg/mL insulin (Sigma-Aldrich, I0516) and the time was recorded as day 0 of differentiation. After two days, medium was replaced with a maintenance medium (DMEM containing 10% fetal bovine serum and 1 μg/mL insulin). Fresh maintenance medium was replaced every 2 days thereafter.

4. Oil Red O Staining

After the porcine preadipocytes were induced to mature adipocytes, remove the complete medium and wash cells 3 times with PBS. The cells were fixed with 4% paraformaldehyde for 1 h at room temperature. Wash cells with 60% isopropanol twice, add oil red 0 solution, stain at room temperature for 30 min, remove the staining solution, then rinsed three times with distilled water and observed under a microscope.

5. qPCR (as Above)

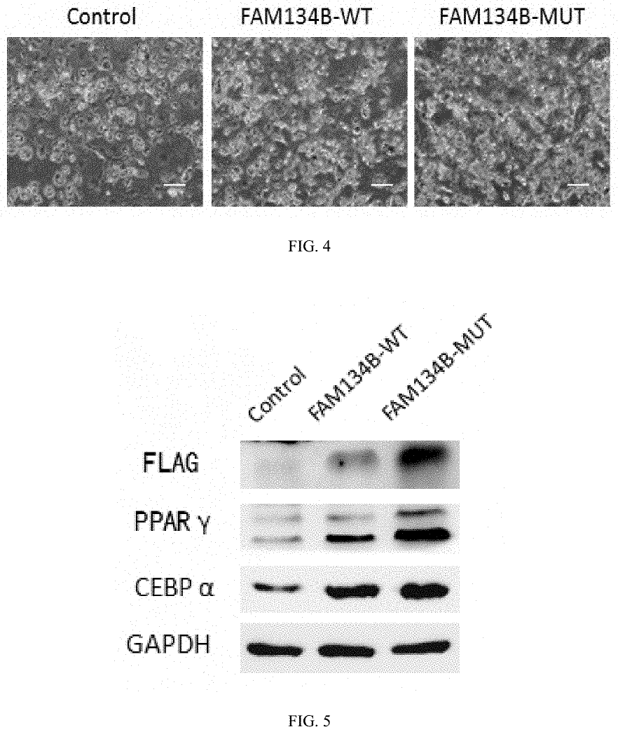

As shown in FIG. 4, mutation of m⁶A site in FAM134B mRNA inhibited adipogenesis and the expression of adipogenic-related genes including PPARγ, FABP4 and C/EBPα.

Embodiment 5: The effect of m⁶A Level of FAM134B mRNA on FAM134B Protein Expression

1. Isolation and Culture of Porcine Preadipocytes (as Above)

2. Porcine Preadipocytes were Transfected with FAM134B-WT or FAM134B-MUT plasmid (as above)

3. Western Blot Analysis

For western blotting analysis, cells were lysed in RIPA buffer containing a protease and phosphatase inhibitor cocktail (Beyotime Biotechnology, Shanghai, China) on ice for 20 min. Samples were then centrifuged at 12,000 rpm for 15 min at 4° C. The same amount of protein was separated by SDS-PAGE, transferred to polyvinylidene difluoride (PVDF) membranes, and incubated in each primary antibody and follow followed by incubations with HRP-conjugated secondary antibodies (HuaBio, Hangzhou, China). The immunoblots were visualized using chemiluminescence (ECL Plus detection system).

Cells were collected and lysed on ice in RIPA buffer for 30 min. The lysates were then centrifuged at 14,000×g for 15 min at 4° C. to remove the insoluble materials. The protein concentrations were measured using a BCA protein assay kit. Equal amounts of protein (30 μg) were heated for 10 min in SDS-PAGE sample buffer. Proteins were separated by SDS-PAGE and then transferred to polyvinylidene difluoride (PVDF) membranes. The membranes were blocked with 5% non-fat milk and 0.1% Tween-20 at room temperature for 1 h and then incubated with a 1:1,000-dilution of primary antibodies overnight at 4° C. After the membranes were washed, they were incubated with a 1:3,000-dilution of HRP-conjugated secondary antibodies at room temperature for 1 h. The immunoblots were visualized using chemiluminescence (ECL Plus detection system).

As shown in FIG. 5, mutation of m⁶A site in FAM134B mRNA promoted FAM134B Protein Expression.

Embodiment 6: The Effect of m⁶A level of FAM134B mRNA on FAM134B mRNA Stability

1. Isolation and culture of porcine preadipocytes (as above)

2. Porcine preadipocytes were transfected with FAM134B-WT or FAM134B-MUT plasmid (as above)

3. mRNA stability analysis

After 24 h of transfection, cells were treated with 5 μg/mL actinomycin D (Sigma, USA) to inhibit mRNA transcription. Samples were collected at 0, 3 and 6 h to assess degradation. The total RNAs were then extracted and

reverse transcribed into cDNA. The mRNA transcript levels of interest were detected by qPCR.

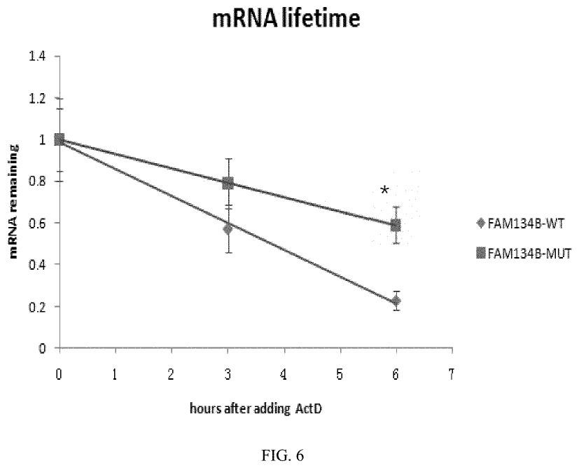

As shown in FIG. 6, point mutation of m⁶A in FAM134B mRNA enhanced mRNA stability of FAM134B.

At last, it should also be noted that the above embodiments are limited specific embodiments of the invention. It is obvious that the present invention is not restricted to the above embodiment. There are many variations. All modifications that can be directly derived or conceived from the present invention by ordinary technician in this field are considered to be under protection.

1. A method of discriminating m⁶A methylation site in FAM134B mRNA which is related to fat deposition in pigs, comprising the steps of:
confirming the m⁶A methylation site in FAM134B mRNA by comparative analyzing m⁶A-seq results of Landrace and Jinhua pigs, corresponding with highly conserved motif RRACH (R=G, A; H=A, C, T) and a prediction website;
altering the m⁶A methylation content in FAM134B mRNA by mutating synonymous codon of FAM134B gene (C1358 to T1358) without changing the amino acid sequence;
designing quantitative real-time PCR (qPCR) primers according to the m⁶A peak region and a control (non-peak) region of the FAM134B mRNA; and
extracting total RNA from the cells and determining the relative m⁶A level of a single gene by protein immunoprecipitation and qPCR technology.

2. The method of claim 1, wherein the comparative analyzing of the difference in m⁶A methylation levels of FAM134B mRNA comprising steps of:
(1) transfecting normal (FAM134B-WT) or mutant (FAM134B-MUT) plasmid in cells for 24 h; extracting total RNA from the cells and fragmentating RNA;
(2) using m⁶A antibody to immunoprecipitate the RNA fragments containing m⁶A modification sites; and
(3) reversing the transcript of the immunoprecipitated RNA into cDNA using qPCR.

3. The method of claim 2, wherein qPCR primers are designed based on total of 21 bases before and after the m⁶A site, A755, and the unmethylated regions, respectively:
pFAM134B-m⁶A-F 5′-CCAAGCAAAGAGAGGCACTCA-3′
pFAM134B-m⁶A-R 5′- CTAACTGGTCTTTGATGGCGG-3′.

4. The method of claim 3, wherein the function of m⁶A modification in FAM134B mRNA is related to adipogenesis, characterized in that:
(1) using normal (FAM134B-WT) or mutant (FAM134B-MUT) plasmid to transfect porcine preadipocytes;
(2) after 48 hours of transfection, inducing the pig preadipocytes into adipocytes differentiation; and
(3) using Oil red O staining and qPCR to validate the effect of FAM134B-WT and FAM134B-MUT on adipogenesis.
